(19) United States
(12) Patent Application Publication
(10) Pub. No.: US 2007/0164232 A1
(43) Pub. Date: Jul. 19, 2007

(54) Devices and Methods for Sanitization

(76) Inventors: Dennis A. Rolleri, Carlstadt, NJ (US); Erin D. London, Gainesville, GA (US); Spyros G. Venduras, New York, NY (US)

Correspondence Address: NORA M. TOCUPS, P.O BOX 698, 140 PINECREST AVE, DECATUR, GA, 30030, US

(21) Appl. No.: 11/625,278

(22) Filed: Jan. 19, 2007

Related U.S. Application Data
(60) Provisional application No. 60/760,763, filed on Jan. 19, 2006.

(51) Int. Cl. G01N 21/00

(52) U.S. Cl. 250/453.11

(57) ABSTRACT

Devices and methods using ultra-violet C (UV-C) radiation and/or its by-product to sanitize and/or deodorize. In use, a device's housing may be placed over the opening of a vessel. The housing may be held in place by a clip(s) that attach(es) to the periphery of the opening. A UV-C radiation source may extend from the bottom of housing into the interior of the vessel. The source may be selectably activated in one or more modes such as a selected time. Upon activation, UV-C radiation and its by-product begin to fill the vessel's interior. Because the opening of the vessel is substantially covered, the radiation and its by-product cannot easily escape. After a time, the radiation and its by-product fill the interior and begin to permeate the interior walls of the vessel, thereby sanitizing and deodorizing the interior and some of the interior walls of the vessel.

### RELATED APPLICATION

This application claims priority to and the benefit of the prior filed co-pending and commonly owned provisional application entitled “Shoe Sanitizer”, which was filed with the United States Patent and Trademark Office on Jan. 19, 2006, which was assigned U.S. Patent Application Ser. No. 60/760,763 and which is incorporated herein by this reference.

### FIELD OF THE INVENTIONS

The inventions relate to ultraviolet C wavelength (UV-C) 100-290 nm, usually 240 nm, sanitizers, and specifically to improvements of those, which may be used in handheld and portable sanitizing applications.

### BACKGROUND

UV-C waves are along the spectrum of UV light. As the wavelength changes, the penetration into human skin changes as well. UV-A is the tanning bed light, primarily with deep penetration to melanin-producing cells (where tans originate), and small amounts remaining in dermis and epidermis to cause burning and DNA damage. UV-B is the sun-burning light, with moderately deep penetration. It reaches to epidermis and dermis layers, there causing burning and DNA damage, and it has minimal penetration to melanin-producing cells. UV-C germicidal rays are superficial, reaching primarily the epidermis and causing burning, but with little penetration to dermis to cause DNA damage, and no penetration to melanin-producing cells. Because UV-C light waves are of a higher energy and shorter wavelength than either UV-A or UV-B, UV-C light waves subsequently destroy cells much more efficiently than either of the other two forms of UV light.

Another advantage of UV-C light is that some wavelengths of UV-C light (such as 185 nm and 254 nm) react with air to form a byproduct that may be used as an effective deodorizer. Specifically, UV-C light of such wavelengths reacts to form ozone gas O₃, which is highly reactive with microbes as well as odor-producing compounds degrading them to less harmful, and more stable products upon contact.

UV-C “germicidal” lights are often installed in commercial applications to reduce microbial contamination of surfaces, air and water. However, prior to now, private, or residential decontamination has been largely limited to chemical cleaners, such as bleach, which may be messy, may produce dangerous fumes and toxins in the environment, and generally are impracticable for edibles, textiles, and other non-durable materials. Increasing use of UV-C germicidal lights is a priority in both commercial and residential settings because of the adverse effects, which chemical cleaners may have on the environment and particularly on wild flora and fauna exposed to polluted waters. Use of UV-C light is also in the interest of public health because of rapidly increasing rates of antibiotic-resistant “super-bugs”. Microbes are not naturally able to develop resistance to UV-C waves.

In the past few years, there has been an increased number of UV-C purification apparatus available to private consumers. The majority of these apparatus are in fixed applications, such as installation into HVAC or plumbing systems and unsuitable for varied usage. A few companies have now developed handheld devices for private use. There are devices available as wands (some available with car lighter attachments) for irradiating large surfaces such as curtains or bedding (exemplars by trade names: Zapitall, Purelight1001, Germinator); there are floor units for irradiating entire rooms (exemplars by trade names: Ultralight 5500 and 4500); there are very small battery-operated units available for travel (exemplars by trade names: FlashUV5, Flash UV7, Ultralite, Germsweeper); and there are even immersible battery-operated devices available for personal water supply purification (See, for example, U.S. Pat. Nos. 5,900,212 and 6,110,424).

Each such apparatus has drawbacks. A wand can only be operated with an operator to hold the wand in the proper direction. A wand generally has a fixed handle, which is often too long and bulky for everyday travel and portability. The floor units in general are too cumbersome for anything but their stated usage on floors. The battery-operated units, restricted in wattage by the size of their batteries, are too weak to efficiently treat an area greater than a few square inches. Microbial kill rate is increased with increasing UV-C wattage, and decreased with decreasing proximity.

In addition to the apparatus described above, there are small, cabinet-like devices, such as shown in U.S. Pat. Nos. 4,806,770 and 6,507,030, for use on discrete objects, but these cannot treat larger objects, or surfaces. None of the above meet the need for compact, portable, convenient, and powerful sterilization such as, for instance, the desire of a business traveler to treat the bedding in an unfamiliar, possibly foreign, hotel room, or to treat the carpet, and bathroom in that room for safer contact with those surfaces; or the desire of a mother to treat the items such as stuffed toys in her child's day-care environment, which would otherwise be left as untreated bacterial reservoirs.

Another problem with the above described apparatus relates to undesired exposure to UV-C light waves. Of the prior art, the majority of cabinet-type and enclosed heating, ventilation and air-conditioning (HVAC) and water treatment applications in general effectively protect humans from UV-C exposure because UV-C light waves do not penetrate most materials. The apparatus with surface treatment options, however, including wands, floor units and other handheld types typically do not provide shielding from either primary rays or reflected rays. Some the manufacturers recommend wearing protective goggles during use.

A specific problem relating to undesired human exposure with respect to the above described prior art apparatus relates to the inadvertent or accidental power-on of such an apparatus. For example, a user may inadvertently leave such an apparatus turned-on longer than intended. As another example, a child might accidentally turn-on an apparatus. Some of the prior art apparatus feature automatic switches, which will turn off the unit after a predetermined length of time. Others have switches which require child-proof or other additional maneuvers to turn on an apparatus. None of these protective measures provide any shielding from the light rays should the apparatus be inadvertently left turned on, or be turned on accidentally in transit, or by a curious child.

Another problem with prior art apparatus relates to the use of light bulbs in such apparatus. UV-C lights generally degrade in efficiency over time, and the speed of that degradation is known to be increased by overheating of the bulb. The degradation leads to prolonged sterilization times and increased necessity for fresh UV-C bulbs, which are rather expensive. No accommodations for this heat degradation effect have been made in any of the prior art listed in the preceding paragraphs. There is a device for maintenance of bulb temperature (U.S. Pat. Nos. 6,832,844 and 6,679,068), however, this maintenance of bulb temperate requires a specially slitted bulb cover, adding to the overall cost of any housing into which it is incorporated.

Advantageously, many if not all of the above drawbacks have been substantially solved by a UV-C sanitizer disclosed in the prior filed co-pending and commonly owned patent application entitled “Methods and Devices for Material Sanitization”, which was filed with the United States Patent and Trademark Office on Jul. 28, 2006, which was assigned U.S. patent application Ser. No. 11/460,966, and which is incorporated herein by this reference. Another device that has solved many, if not all, of the above drawbacks is described in the prior filed co-pending and commonly owned patent application entitled “Apparatuses and Methods for Sanitization”, which was filed with the United States Patent and Trademark Office on Sep. 15, 2006, which was assigned U.S. patent application Ser. No. 11/532,435, and which is incorporated herein by this reference.

The advantages of the UV-C sanitizers in the previous paragraph prove useful in many situations, but there remain products and areas where more problem specific sanitizers would be useful. For example, items that have relatively small interior areas may benefit from a sanitizer that is sized to the specific interior areas. As another example, items and areas that call for sanitization because of microbial contamination (and other reasons) often also have a bad or unpleasant odor. Thus, there is a need for devices and methods that may deodorize as well as sanitize relatively small interior areas of things and objects. Yet, it would be undesirable to require a user to purchase different sanitizers for different items and purposes. Thus, there is a need for devices and methods that may deodorize as well as sanitize relatively small interior areas of things and objects and that may be used in other circumstances as well.

### SUMMARY

Generally stated, the inventions relate to devices and methods using an ultra-violet C (UV-C) radiation for sanitization of objects and areas and that also may be used for deodorizing these objects and areas.

Advantageously, the inventions allow for the increased use of UV-C germicidal lights in all types of settings in place of chemical cleaners and their adverse effects. Increased use of UV-C radiation is in the interest of public health because of rapidly increasing rates of antibiotic-resistant “super-bugs”. Microbes are not naturally able to develop resistance to UV-C waves.

A particular advantage of the inventions is that they allow for the increased use of UV-C germicidal lights to substantially deodorize as well as substantially sanitize objects and areas.

A first exemplary embodiment of the inventions is referred to as a sanitizing and deodorizing device. The device includes a housing shaped generally to substantially cover the opening of a vessel such as a shoe, a thermos, and other items. The housing may include a clip, clips, or other elements that may be attached to a peripheral point of the opening of the vessel. The clip, clips, or other elements may facilitate the positioning of the housing to cover the opening of the vessel and/or to hold the housing in place over the opening of the vessel.

An alternative embodiment of the inventions may provide a collar to be placed around the periphery of the housing. When the housing with the collar is placed over the opening to the vessel, the collar may aid in completely covering the opening. Alternatively, the collar may be used with the housing of the sanitizing and deodorizing device when it is used with vessels that have openings that cannot be covered by the housing alone.

Referring again to the first exemplary embodiment, the housing includes a bottom facing the interior of the vessel. The bottom of the housing may have a surface capable of reflecting any UV-C light that encounters the bottom back into the interior of the vessel. An ultra-violet C (UV-C) light source is attached to the bottom of the housing. The UV-C light source may be extendible into the interior of the vessel.

A power supply included in the housing powers the UV-C light source. The housing also includes a selectable power-on mechanism for activating the UV-C light source when the power-on mechanism is selected. The power-on mechanism may be configured so that its selection allows for continuous activation of the UV-C light source and/or activation of the source for a period(s) of time. Advantageously, the UV-C light sanitizes the interior of the vessel when the UV-C light source is powered-on, and the UV-C light causes ozone to be produced in the interior of the vessel, thereby deodorizing the interior of the vessel as well as sanitizing it.

The housing of this first exemplary embodiment also may include a mode switch connected to the power-on mechanism. A first activation of the mode switch causes the power-on mechanism to activate the UV-C light source in a first mode such as operation for a first period of time. A second activation of the mode switch causes the power-on mechanism to activate the UV-C light source in a second mode such as operation for a second period of time. The first activation of the mode switch also may cause a first indicator to indicate the activation of the UV-C light source in the first mode. Similarly, the second activation of the mode switch may cause a second indicator to indicate the activation of the UV-C lights source in the second mode.

Another exemplary embodiment of the inventions provides a footwear sanitizer. This sanitizer may include a housing shaped substantially to cover the opening of an example of footwear such as an athletic shoe or sneaker. An ultra-violet C (UV-C) light source is mounted to the bottom of the housing so that the position of UV-C light source with respect to the bottom of the housing may be adjusted. Adjusting the UV-C light source away from the bottom of the housing (when the housing covers the opening) causes the UV-C light source to extend into at least a part of the interior of the example of footwear. Advantageously, this extendibility facilitates introduction of UV-C light and resulting by-product into the interior of the example of footwear.

In this exemplary embodiment, the bottom of the housing includes a well for receiving at least part of the UV-C light source when the UV-C light source is adjusted to a position substantially parallel to the bottom of the housing. Advantageously, the fit of the UV-C light source (at least partially) into the well in the bottom of the housing makes for a relatively compact and easy to transport footwear sanitizer.

The housing of the exemplary embodiment includes an on/off button connected to a power on/off mechanism. The power on/off mechanism is connected to a power source for powering the UV-C light source. The power source may be external and/or internal to the housing. A jack may be provided on the housing to facilitate connection of the footwear sanitizer to an external power source.

The power on/off mechanism of the footwear sanitizer also activates or deactivates the UV-C light source in response to selection of the on/off button. A timing selector may be disposed on the housing so that a user may select activation of the UV-C light for a period of time. The housing also may include an indicator to indicate activation and/or de-activation of the UV-C light source, and/or to indicate the activation and/or de-activation of the UV-C light source in a particular mode of activation as may be selected from more than one modes of activation.

An advantage of the exemplary embodiment is that the UV-C light emitted into the interior of the example of footwear results in deodorizing gas produced in the interior. In addition, the substantial covering of the opening of the footwear causes entrapment of the deodorizing gas. Thus, the deodorizing gas is produced in the interior of the example of footwear and is contained therein so that deodorizing of the interior is facilitated as compared to what deodorizing may occur if the opening of the footwear is not covered or not substantially covered.

Yet another exemplary embodiment of the inventions provides a sanitizer including a body having an ultra-violet C (UV-C) radiation source pivotally attached to a point on the exterior of the body. The UV-C radiation source is pivotably positionable within at least an approximately 120 degree arc with respect to a horizontal at the attachment point of the UV-C radiation source to the body.

In this exemplary embodiment, the body includes a control mechanism for causing operation of the UV-C radiation source for a period of time in response to receipt of an indication for such operation. The body includes an indicator selectable to provide the indication for the operation of the UV-C radiation source for the period of time to the control mechanism. The body also is connected to a power source connected to the control mechanism to power the operation of the UV-C radiation source.

In sum, the inventions provide for UV-C sanitizers that may prove useful in many situations for their sanitization and/or their deodorizing features. For example, exemplary embodiments of the inventions may be used with items that have enclosed or interior areas, and may be particular useful with items whose interiors are relatively small. As another example, exemplary embodiments may be used to deodorize items, objects, and areas that have a bad or unpleasant odor. Advantageously, a user is not required to purchase different sanitizers and deodorizers for different items, areas and purposes. The exemplary embodiments provide for devices and methods that may deodorize as well as sanitize items and areas in general, and the interiors of such items and areas.

### BRIEF DESCRIPTION OF THE DRAWINGS

FIG. 1 is a side perspective view illustrating an exemplary embodiment of the inventions as it may be used with an athletic shoe.

FIG. 2 is also a side perspective view illustrating an exemplary embodiment of the inventions as it may be used with an athletic shoe, but with a portion of the athletic shoe cutaway to show part of the interior of the athletic shoe.

FIG. 3 is a view of the bottom of an exemplary embodiment of the inventions.

FIG. 4 is a side view of an exemplary embodiment of the inventions.

### DETAILED DESCRIPTION

The inventions are described in detail below sufficiently for a person skilled in the art to make or use the inventions. The inventions are described by reference to exemplary embodiments including devices and methods. The inventions, however, should not be limited to these embodiments, but may also include other elements, actions, apparatuses, and methods (not specifically described) in accordance with the inventions.

Generally stated, the inventions relate to devices and methods using an ultra-violet C (UV-C) radiation for sanitization of objects and areas and also may be used for deodorizing these objects and areas. The deodorizing feature may be implemented by providing that the UV-C radiation that is emitted from the UV-C radiation source is of the appropriate wavelength (for example, 185 or 254 nm) that results in the generation of O₃ (which may be referred to herein as ozone, deodorizing by-product, deodorizing gas, and similar terms), which is a deodorizer.

Still stated generally, the inventions may provide a device that includes a housing with an attached UV-C radiation source. In use, the device's housing may be placed over the opening of a vessel so as to substantially cover the opening. The housing may be held in place by a clip(s) that attach(es) to the periphery of the opening. The UV-C radiation source may extend from the bottom of housing into the interior of the vessel. The source may be selectably activated in one or more modes such as for a selected time. Upon activation, UV-C radiation and its by-product begin to fill the vessel's interior. Because the opening of the vessel is substantially covered, the radiation and its by-product cannot easily escape. After a time, the radiation and its by-product fill the interior and begin to permeate the interior walls of the vessel, thereby sanitizing and deodorizing the interior and some of the interior walls of the vessel. UV-C radiation also may be referred to herein as UV-C light or UV-C waves, or similar terms.

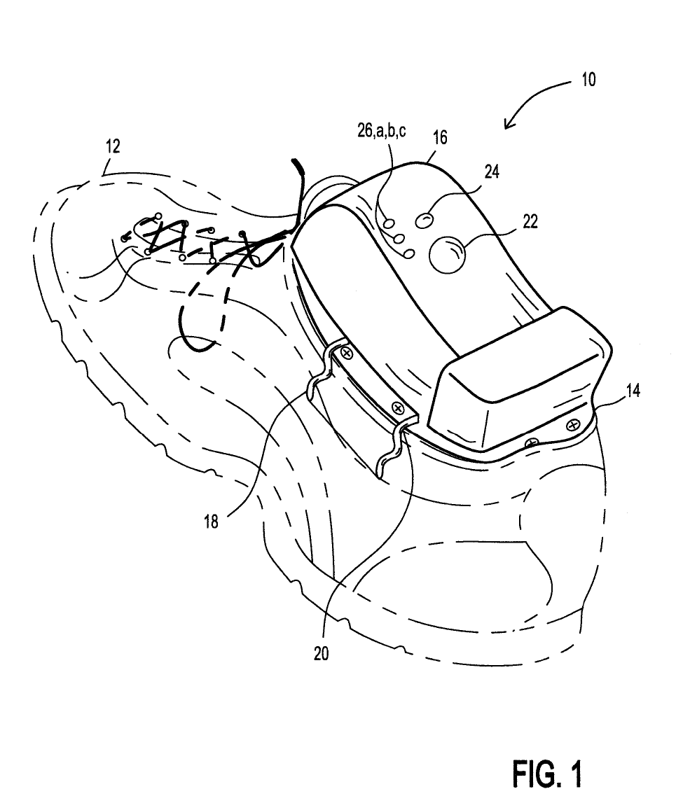

FIG. 1 shows an exemplary device 10 according to the inventions as it may be used with a vessel 12, which in this case is an athletic shoe (also referred to as a sneaker). The term “vessel” as used herein may include generally items that have an interior accessible by at least an opening that may be covered to contain the UV-C radiation and its ozone by-product so as to accomplish sanitization and/or deodorizing. Vessels may include footwear, containers such as coolers, thermoses, vases, garbage cans, diaper hampers, enclosed spaces such as cubby holes or small contained areas, other items like garment bags, gym bags, purses, briefcases, luggage, and the like. A vessel having more than one opening can be used with the exemplary embodiments, but substantially covering all or most of the openings of such a vessel facilitates containment of the radiation and ozone by-product to better effectuate sanitization and deodorizing of the interior of the vessel.

FIG. 1 illustrates that the exemplary device 10 substantially covers the generally oval opening 14 of the shoe 12 by which a user inserts his or her foot into the shoe 12. In particular, the housing 16 of the device 10 substantially covers the opening 14 by being generally oval in shape and sized adequately with respect to the shoe's opening 14. As will be seen below from the discussion of alternative embodiments, the housing 16 does not necessarily have to be oval nor sized just to cover a shoe's opening, but may be variously sized (and even include adjustable sizing) so as to substantially cover an opening in a vessel with which the device 10 is used.

In the example illustrated in FIG. 1, the athletic shoe 12 is shown as laced up. Such lacing may facilitate the sanitization and deodorizing because it allows for better containment of the UV-C radiation and ozone by-product in the interior of the shoe 12 than an unlaced shoe. Nonetheless, the exemplary device 10 may be used with unlaced shoes (or otherwise “undone” shoes such as unbuckled, unzipped, etc.), but the sanitization and/or deodorizing may not be as quickly nor as effectively achieved as when the shoe is laced.

In this example, the device 10 includes attachment mechanisms in the form of clips (only one clip and related elements are illustrated in FIG. 1) 18. The clips 18 are attached to respective opposite sides of the device's housing 16 and extend to attach onto respective peripheral points 20 of the opening 14 of the shoe 12. One function of the clips 18 is to position the device 10 over the opening 14 of the 12, and another function is to hold the device 10 in place over the opening 14. Yet another function that the clips 18 may serve is to better effect the seal or covering of the opening 14 by the device 10. The clips 19 may better effectuate the covering of the opening 14 by the housing by drawing the sides of the shoe 12 closer towards the center of the opening 14. This may reduce the size of the opening and/or change its general oval shape.

FIG. 1 further illustrates that the device's housing 16 may include various features with respect to activation and operation of the device 10. In this example, the device includes an on-off button 22 that may be used to turn-on or activate (and/or turn-off or de-activate) the device 10. The on-off button 22 may be or may be part of a selectable power-on mechanism (also referred as a power on/off mechanism and/or control mechanism) for activating or de-activating of a UV-C light source of the device 10 in response to pushing of the button 22.

The embodiment illustrated in FIG. 1 includes other features relating to the activation of the UV-C radiation source, but an alternative embodiment may include only the on-off button 22 (and related mechanism) so that the only alternatives are to turn the UV-C radiation source of the alternative embodiment on or to turn it off. Yet another alternative embodiment may provide that repeated pushing of an on-off button may result in changing the mode or modes of operation of the alternative device. For example, pushing the on-off button once may cause the alternative device to activate the UV-C radiation source for a first period of time—say 5 minutes. Pushing the on-off button twice in relatively quick succession may cause the alternative device to activate for a second period of time—say 10 minutes. Similarly, pushing the on-off button three times in relatively quick succession may cause the alternative device to activate for a third period of time—say 15 minutes. Other ways to implement one or more modes of operation by using only an on-off button will occur to those skilled in the art.

The exemplary device 10 of FIG. 1 also includes a mode switch (also referred to as a mode selector) 24 that may enable a user to implement or change a mode of operation (also referred to as modes of activation) of the device. A mode of operation of a device according to the inventions may refer to various ways of using, activating, or operating the device. For example, a mode of operation may refer to the length of time that the UV-C radiation source is activated to emit UV-C radiation. Thus, a first mode of operation may be a first period of time, a second mode of operation may be a second period of time, etc. As another example, a mode of operation may refer to the wavelength, power, and/or other features of the UV-C radiation that may be varied. Of course, a mode of operation may specify a period of time as well as another particular feature such as power level and/or other combinations of features for mode operation. The mode switch 24 is configured, preferably, to conform to the number of modes in which the device 10 may operate and allows a user to select a particular one of the available modes. Obviously, the mode switch 24 may be implemented in other forms such as a push-button, dial, etc.

Assume that the exemplary device 10 illustrated in FIG. 1 may be operated in three modes with each mode corresponding to activation of the UV-C radiation source for a respective amount of time. Advantageously, the exemplary device 10 includes three indicators 26a-c on its housing 16 to provide information on the activation of the device 10 in a particular mode. For example, if the device is operating in the first mode, such as activation of the UV-C radiation source for five minutes, then the first indicator 26a may be illuminated. When the UV-C radiation source is de-activated, the first indicator may be dimmed. Similarly, the second indicator 26b may be illuminated only when the device 10 is operated in the second mode, and the third indicator 26c may be illuminated only when the device 10 is operated in the third mode. The exemplary device 10 uses separate indicators for each mode, but other ways of showing operation of the device in different modes may be provided in addition or in the alternative.

In the exemplary device 10, the on-off functions, the activation/de-activation of the UV-C radiation, and the illumination/dimming of the indicators are carried out by a power-on mechanism (also referred as a power on/off mechanism and/or control mechanism) that may be implemented with electronic circuitry enclosed within the housing 10 and not shown in FIG. 1 (or otherwise implemented), or otherwise. The exemplary device 10 may be powered by an interior power source or supply (not shown in FIG. 1) such as a battery, etc., and/or may be powered by an appropriate connection (including conversion elements such as an AC power converter) to an exterior or external power source or supply such as a residential or commercial electrical utility outlet. Thus, the exemplary device 10 may include a jack or other port to allow the connection of the device 10 to such an external power source.

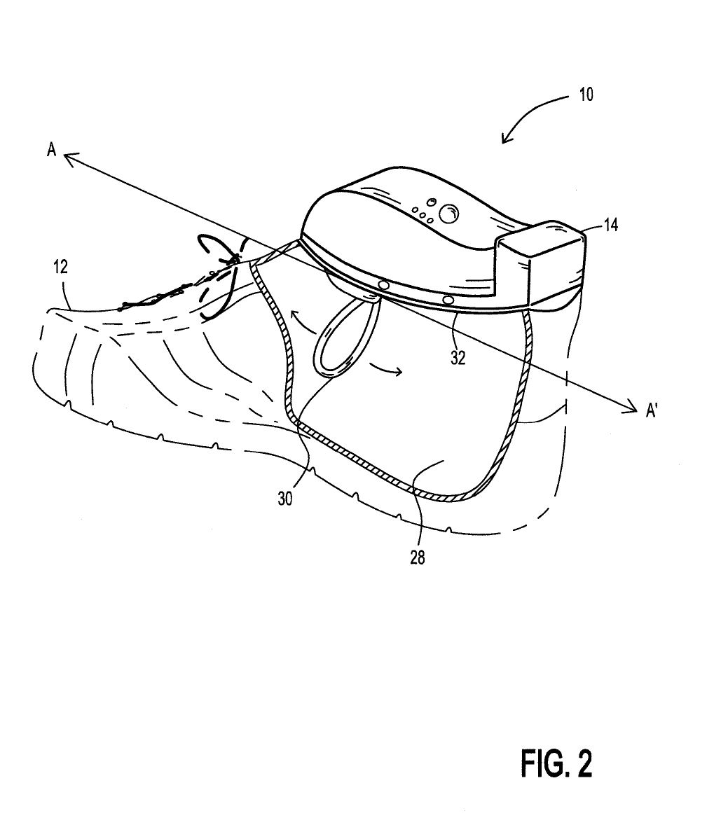

FIG. 2 illustrates (by partial cut away of the outside of the shoe 12 to show its interior 28) that the exemplary device 10 includes a UV-C radiation source 30 attached to the bottom 32 of the housing 14. Generally stated, the bottom 32 of the housing 14 faces the interior 28 of the vessel or shoe 12 in this case. Unlike the illustration in FIG. 2, the UV-C radiation source 30 may be attached (so that it cannot be adjusted or otherwise) so that it lies in parallel with or is part of the bottom 32 of the housing 14. In fact, a positionably adjustable UV-C radiation source may be used in such a bottom parallel or part of the bottom position. Nonetheless, using the UV-C radiation source in such a bottom parallel or part of the bottom position (whether positionably adjustable or not) may (presumably negatively) effect the time and/or effectiveness of the sanitization and/or deodorizing.

In FIG. 2, the UV-C radiation source 30 is shown as a somewhat flattened oval or generally U-shaped tubular light fixture, but this is variable. Moreover, the UV-C radiation source 30 may include more than one light fixture, and/or other mechanism that allows for emission of UV-C radiation, and for deodorizing purposes, for emission of UV-C radiation of the appropriate wavelength (whether 185, 254 nm or otherwise) to result in ozone creation.

Generally, the UV-C radiation source 30 is sized and configured so as to extend into or to be extendible into and fit within the interior 28 of the shoe 12. The source 30 may be positionably adjustable so as to more effectively and quickly deliver emitted UV-C radiation to all or most parts of the interior 28 of the shoe 12. In the example of FIG. 2, the UV-C radiation source 30 is positionably adjustable by a pivoting connection or attachment to the bottom 32 of the housing 14 of the exemplary device 10. This pivoting connection may be accomplished in any appropriate matter. This particular pivoting connection may allow for the positioning of the UV-C radiation source 30 anywhere along an arc of at least 120 degrees with respect to the horizontal being a point of attachment of the source 30 to the bottom 32 of the shoe 12. In this example, the horizontal is illustrated by the imaginary line shown as A-A′ in FIG. 2. Alternative embodiments may provide that a UV-C radiation source may be positioned only at certain points along an arc such as discussed, rather than being positionable at points along the arc.

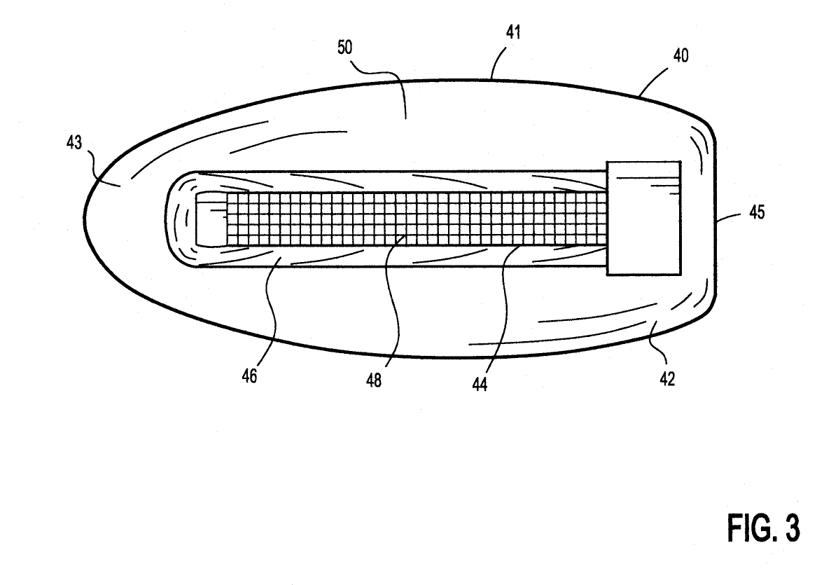

As also shown in FIG. 2, the UV-C radiation source 30 is pivotally attached to the bottom 32 of the housing 14 nearer to the front part of the exemplary device 10 (with the front being the part of the device closest to the area of the shoe covering the toes) than the back part. Alternative embodiments may provide for a similar pivotal attachment (or other attachment) to other parts of the bottom 32 of the housing 14. For example, a UV-C radiation source may be connected near the back bottom (see discussion in connection with FIG. 3) or central bottom of the housing of an alternative device.

FIG. 3 illustrates an alternative embodiment of a device 40 according to the inventions. The housing 41 of the alternative device 40 has a generally flattened oval shape or periphery with the front 43 of the device 40 being more pointed than the back 45 so as to better suit the general shape of an opening in a shoe. FIG. 3 specifically is a view of the bottom 42 of the device 40. In the exemplary alternative device 40, a wand-like UV-C radiation source 44 is pivotably attached towards the back 45 of the bottom 42 of the device 40 so as to extend the source 44 into the interior of a shoe (or other object).

In the example of FIG. 3, the UV-C radiation source 44 is shown in a “closed” position by being at least partially positioned in a well 46 set into the bottom 42 of the device 40. The well 46 may be configured so as to allow the source 44 to completely fit within the well 46 in the closed position so that none of the source 44 protrudes beyond the bottom 42 of the device 40. Whether completely or partially fit into the well 46, the closed position of the UV-C radiation source 44 allows the device 40 to provide an overall compact profile to facilitate ease of storage, transportation, and other actions.

FIG. 3 also illustrates that the UV-C radiation source 44 may be covered by a shield 48. In alternative embodiments, the UV-C radiation source may be only partially covered by a shield (and/or other protective mechanism). Advantageously, the shield 48 allows for the emission of the UV-C radiation, but does not allow for broken glass, or other elements to pass. Thus, the shield 48 may increase safety in use of a device according to the inventions.

FIG. 3 further shows another feature of the inventions that may provide for greater effectiveness or speed of use of a device to achieve adequate sanitization and/or deodorizing. This feature is the coating (or other covering) of the bottom 42 of the device 40 with material 50 that reflects UV-C radiation. In this example, all of the bottom 42 of the device 40 is covered with the reflective material 50, but other embodiments may provide for partial covering (or no covering). The reflective material 50 causes UV-C radiation that encounters the reflective material to bounce back into the interior of the vessel (such as a shoe) so as to further contribute to the sanitization and/or deodorizing of the interior. This further contribution may speed and/or add to the effectiveness of the sanitization and/or deodorizing. An example of a reflective material that may be used is flash chrome plating.

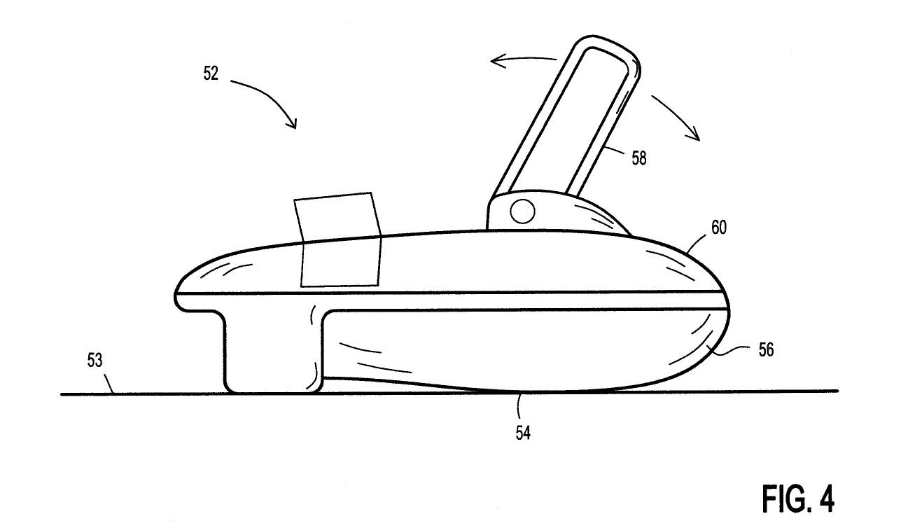

FIG. 4 shows yet another way in which an another exemplary embodiment 52 of the inventions may be used. FIG. 4 is a side view of the exemplary device 52. As shown in FIG. 4, the device 52 is placed on a flat surface 53, and is turned upside down from the positions of the devices shown in the previous figures. In other words, in FIG. 4, the device 52 is resting on the top 54 (where the controls such as on/off, mode switch, etc. may be disposed) of its housing 56. If the controls are disposed on the top 54 of the device 52, then they may be configured so that resting the device 52 upside on its top 54 does not engage and/or effect the controls.

The exemplary device 52 includes a UV-C radiation source 58 pivotably attached to the bottom 60 of its housing 56. The UV-C radiation source 58 extends away generally from the bottom 60 of the housing 56, and in this case, away from the flat surface 53 upon which the device 52 rests. The UV-C radiation source 58 may be positioned along an arc of at least 120 degrees with respect to the horizontal at the position of its attachment to the bottom 60 of the housing 56. Other attachments of a UV-C radiation source may be provided in alternative embodiments.

Myriad uses of the exemplary device 52 in the position illustrated in FIG. 4 may be implemented. For example, a user may sanitize and/or deodorize (at least partially) the interior of his or her locker by positioning the exemplary device 52 as illustrated in FIG. 4. Depending on its size, the interior of the locker may be sufficient to contain the UV-C radiation and resulting ozone by-product so as to accomplish at least partial sanitization and/or deodorizing. To facilitate the use of the exemplary device 52 in this position, the device 52 may be provided with a timer allowing prolonged activation of UV-C radiation source so as to more effectively sanitize and/or deodorize. As another example, a user may sanitize and/or deodorize the interior of a garbage can by placing the exemplary device 52 in the upside down position in the inside bottom of the garbage can. As yet another example, a user may sanitize and/or deodorize the interior of a garment bag or similar item such as a gym bag by placing the exemplary device 52 in the upside down position in the inside bottom of the bag. Other uses may be made with refrigerators, dishwashers, washing machines. Of course, the devices according the inventions may be used with the previously mentioned examples as positioned as shown in the previous illustrations or otherwise.

From the above, the reader will understand that devices and methods according described herein according to the inventions may serve well in travel and in other uses because they are compact, portable and versatile devices that can be used to sanitize and/or deodorize almost any material in almost any situation without damage generally to the material, the environment, and/or the users themselves.

From the foregoing description of the exemplary embodiments of the inventions and operation thereof, other embodiments will suggest themselves to those skilled in the art. Therefore, the scope of the inventions is to be limited only by the claims below and equivalents thereof.

We claim:

1. A sanitizing and deodorizing device, comprising:
a housing shaped generally to substantially cover the opening of a vessel, the housing having a bottom facing the interior of the vessel;
an ultra-violet C (UV-C) light source attached to the bottom of the housing;
the UV-C light when emitted by the UV-C light source into the interior of the vessel resulting in ozone production in the interior of the vessel;
the housing including a power supply to power the UV-C light source; and
the housing including a selectable power-on mechanism for activating the UV-C light source in response to selection of the power-on mechanism,
whereby UV-C light sanitizes the interior of the vessel when the UV-C light source is powered-on, and the ozone resulting from the emission of the UV-C light substantially deodorizes the interior of the vessel.

2. The sanitizing and deodorizing device of claim 1, wherein the bottom of the housing comprises a surface capable of reflecting the UV-C light.

3. The sanitizing and deodorizing device of claim 1, wherein the UV-C light source is extendible into the interior of the vessel.

4. The sanitizing and deodorizing device of claim 1, further comprising at least a clip to attach the housing to the vessel.

5. The sanitizing and deodorizing device of claim 4, wherein the clip attaches to the vessel at a generally peripheral point of the opening of the vessel covered by the housing.

6. The sanitizing and deodorizing device of claim 1, further comprising an attachable collar for attaching around the periphery of the housing to completely cover the opening of the vessel, or to cover a larger opening of another vessel.

7. The sanitizing and deodorizing device of claim 1, wherein the selectable power-on mechanism activates the UV-C light source for a period of time in response to the selection of the power-on mechanism.

8. The sanitizing and deodorizing device of claim 1, wherein the housing comprises a mode switch connected to the power-on mechanism with a first activation of the mode switch causing the power-on mechanism to activate the UV-C light source in a first mode, and a second activation of the mode switch causing the power-on mechanism to activate the UV-C light source in a second mode.

9. The sanitizing and deodorizing device of claim 8, wherein the first mode comprises the activation of the UV-C light source for a first period of time; and
wherein the second mode comprises the activation of the UV-C light source for a second period of time.

10. The sanitizing and deodorizing device of claim 8, wherein the first activation of the mode switch causes a first indicator to indicate the activation of the UV-C light source in the first mode.

11. A footwear sanitizer, comprising:
a housing shaped substantially to cover the opening of an example of footwear;
an ultra-violet C (UV-C) light source mounted to the bottom of the housing so that the position of UV-C light source with respect to the bottom of the housing may be adjusted, and so that adjusting the UV-C light source away from the bottom of the housing when the housing covers the opening causes the UV-C light source to extend into at least a part of the interior of the example of footwear;
the bottom of the housing including a well for receiving at least part of the UV-C light source when the UV-C light source is adjusted to a position substantially parallel to the bottom of the housing; and
the housing including an on/off button connected to a power on/off mechanism, which is connected to a power source for powering the UV-C light source, and which activates or deactivates the UV-C light source in response to selection of the on/off button.

12. The footwear sanitizer of claim 11, wherein the power source comprises an external power source.

13. The footwear sanitizer of claim 12, wherein the housing comprises a jack for connection to the external power source.

14. The footwear sanitizer of claim 11, wherein the power source comprises a source interior to the housing.

15. The footwear sanitizer of claim 11, wherein the housing comprises a timing selector allowing for activation of the UV-C light source for a selectable period of time.

16. The footwear sanitizer of claim 11, wherein the housing comprises an indicator to indicate activation of the UV-C light source and to indicate de-activation of the UV-C light source.

17. The footwear sanitizer of claim 11, wherein the housing comprises an indicator system to indicate activation of the UV-C light source in a particular mode of more than one modes of activation.

18. The footwear sanitizer of claim 11, wherein the UV-C light emitted into the interior of the example of footwear results in deodorizing gas produced in the interior; and
wherein the substantial covering of the opening of the example of footwear causes entrapment of the deodorizing gas, thereby substantially deodorizing the interior of the example of footwear.

19. A sanitizer, comprising:
a body having an ultra-violet C (UV-C) radiation source pivotally attached to a point on the exterior of the body, the UV-C radiation source being pivotably positionable an arc of at least approximately 120 degrees with respect to a horizontal at the attachment point of the UV-C radiation source to the body;
the body including a control mechanism for causing operation of the UV-C radiation source for a period of time in response to receipt of an indication for the operation for the period of time;
the body including an indicator selectable to provide the indication for the operation of the UV-C radiation source for the period of time to the control mechanism; and
the body being connected to a power source connected to the control mechanism to power the operation of the UV-C radiation source.

20. The sanitizer of claim 19, wherein the UV-C radiation emitted by the UV-C radiation source results in ozone production in an area, thereby deodorizing the area.
